(19) United States
(12) Patent Application Publication
(10) Pub. No.: US 2025/0226193 A1
(43) Pub. Date: Jul. 10, 2025

(54) DEVICE FOR PROCESSING MASS SPECTROMETRY DATA

(71) Applicant: SHIMADZU CORPORATION, Kyoto (JP)

(72) Inventor: Yoshito KAWAKITA, Kyoto-shi (JP)

(73) Assignee: SHIMADZU CORPORATION, Kyoto (JP)

(21) Appl. No.: 19/012,772

(22) Filed: Jan. 7, 2025

(30) Foreign Application Priority Data
Jan. 9, 2024 (JP) 2024-001007

(51) Int. Cl. H01J 49/00; G01N 33/487

(52) U.S. Cl. CPC H01J 49/0036; G01N 33/48792; H01J 49/005

(57) ABSTRACT

A device for processing mass spectrometry data (40) includes: a storage section (41) which holds information in which reagent-identifying information is related to the mass-to-charge ratio of an ion to be generated from the reagent; a display section (60); a mass-spectrum-data input receiver (42); a reagent-information input receiver (45) configured to receive an input of the reagent identification information of a reagent added to a sample; a peak locator (46) configured to compare the inputted reagent identification information with the information stored in the storage section, and to locate a peak associated with an ion generated from the reagent; and a display processor (47) configured to create a mass spectrum from the mass spectrum data, and to display, on the display section, the mass spectrum in such a manner that the peak located by the peak locator is shown in a form distinguishable from other peaks.

### TECHNICAL FIELD

The present invention relates to a device for processing mass spectrometry data.

### BACKGROUND ART

One type of device for the qualitative and/or quantitative determination of metabolites contained in a sample originating from a living organism is a gas chromatograph mass spectrometer. For an analysis of a metabolite using a gas chromatograph mass spectrometer, the process of trimethylsilylation, i.e., the derivatization of the metabolite through the addition of a trimethylsilyl group (Si(CH₃)₃) to the metabolite in the sample, is often performed for increasing the volatility of the metabolite. The sample containing the trimethylsilylated metabolite is vaporized within a sample vaporization chamber of the gas chromatograph, separated into components by a column, and introduced into the mass spectrometer, in which each component is ionized and subjected to a mass spectrometric analysis (for example, see Patent Literature 1).

In the qualitative determination of the metabolite contained in the sample by a mass spectrometer, a mass spectrum is acquired by a scan measurement and compared with the mass spectrum of a known compound recorded in a compound database. Based on the degree of similarity of the two mass spectra, the qualitative determination of the metabolite is performed. In the quantitative determination of the metabolite contained in the sample, a selected ion monitoring (SIM) measurement or multiple reaction monitoring (MRM) measurement is performed. For the SIM or MRM measurement, a target ion is previously designated for each metabolite which is a target of the measurement, and a calibration curve is created beforehand from the area value or intensity value of a peak of the target ion obtained by a measurement of a standard sample. In an actual measurement of a sample, the quantity of the metabolite is determined from the measured value of the area or intensity of the peak of the target ion with reference to the calibration curve.

### CITATION LIST

### Patent Literature

Patent Literature 1: JP 2018-169376 A

### SUMMARY OF INVENTION

### Technical Problem

Derivatizing a sample means that an ion originating from the derivatization reagent is generated in addition to the ions originating from the metabolite when the ionization is performed in the mass spectrometer. Therefore, not only the peaks of the ions originating from the metabolite but also the peak of the ion originating from the derivatization reagent will appear in the mass spectrum of the metabolite.

In the qualitative determination of a metabolite contained in a sample, when a mass spectrum acquired by a measurement of the sample is compared with the mass spectra of known compounds recorded in the compound database, a plurality of candidate compounds which are close to each other in degree of similarity may possibly be extracted. An analysis operator skilled in metabolite analysis can exclude ions originating from the derivatization reagent and perform the qualitative determination of the metabolite by selecting appropriate compounds based on the degree of similarity in the mass distribution of the other ions. In contrast, an analysis operator who is not skilled in metabolite analysis may possibly make a false-positive identification by performing the qualitative determination based on the degree of similarity in the mass distribution of the ions originating from the derivatization reagent.

As regards the target ion used in an SIM or MRM measurement of a metabolite, a scan measurement is performed to acquire a mass spectrum, and an ion detected with a high intensity is designated as the target ion. Once again, an analysis operator who is not skilled in metabolite analysis may possibly designate, as the target ion, an ion originating from the derivatization reagent, without knowing that the ion has originated from the reagent. Using an ion originating from a derivatization reagent as a target ion in an SIM or MRM measurement leads to a false-positive detection of another metabolite derivatized in a similar manner to the target metabolite or a compound originating from the reagent or other substances, so that the qualitative/quantitative determination of the target metabolite cannot be correctly performed.

Although the examples described thus far have been concerned with the case of using a derivatization reagent for increasing the volatility of a metabolite in a gas chromatograph, the previously described problem can similarly occur in various situations in which a mass spectrometric analysis is performed for a sample to which a reagent is added, as in the case of using a reagent for increasing the ionization efficiency in the mass spectrometer.

The problem to be solved by the present invention is to provide a technique which enables an analysis operator to easily recognize a peak associated with an ion originating from a reagent in a mass spectrum acquired by a mass spectrometric analysis of a sample to which a reagent is added, regardless of the level of skill of the analysis operator.

### Solution to Problem

A device for processing mass spectrometry data according to the present invention developed for solving the previously described problem includes:
a storage section which holds information in which reagent identification information identifying a reagent is related to the mass-to-charge ratio of an ion to be generated from the reagent;
a display section;
a mass-spectrum-data input receiver configured to receive an input of mass spectrum data acquired by a mass spectrometric analysis of a sample;
a reagent-information input receiver configured to receive an input of the reagent identification information identifying a reagent added to the sample in the mass spectrometric analysis;
a peak locator configured to compare the reagent identification information received by the reagent-information input receiver with the information stored in the storage section, and to locate, among the peaks included in the mass spectrum data received by the mass-spectrum-data input receiver, a peak associated with an ion generated from the reagent; and
a display processor configured to create a mass spectrum from the mass spectrum data, and to display, on the display section, the mass spectrum in such a manner that the peak located by the peak locator is shown in a form distinguishable from other peaks.

### Advantageous Effects of Invention

In the device for processing mass spectrometry data according to the present invention, when an analysis operator has entered mass spectrum data acquired by a mass spectrometric analysis of a sample as well as reagent identification information which identifies the reagent added to the sample, the device creates a mass spectrum from the mass spectrum data and displays it on the display section in a form in which a peak associated with an ion originating from the reagent is distinguishable from other peaks. Therefore, the user can easily recognize a peak associated with an ion originating from the reagent in the mass spectrum, regardless of the level of skill of the user.

### BRIEF DESCRIPTION OF DRAWINGS

FIG. 1 is a configuration diagram of the main components of a gas chromatograph mass spectrometry system including one embodiment of the device for processing mass spectrometry data according to the present invention.

FIG. 2 is a flowchart illustrating the procedure for determining a target ion in an SIM measurement using the gas chromatograph mass spectrometry system according to the present embodiment.

FIG. 3 is an example of a mass spectrum in which the peaks associated with ions originating from a reagent are highlighted in the present embodiment.

FIG. 4 is another example of a mass spectrum in which the peaks associated with ions originating from a reagent are highlighted in the present embodiment.

FIG. 5 is an example of a mass spectrum in which the peaks associated with ions originating from a reagent are displayed in an obscure form in the present embodiment.

FIG. 6 is an example of a mass spectrum in which the peaks associated with ions originating from a reagent are accompanied by the structural formula of the ions in the present embodiment.

FIG. 7 is an example of a mass spectrum in which two peaks having large mass-to-charge ratios are highlighted in the present embodiment.

### DESCRIPTION OF EMBODIMENTS

One embodiment of the device for processing mass spectrometry data according to the present invention is hereinafter described with reference to the drawings.

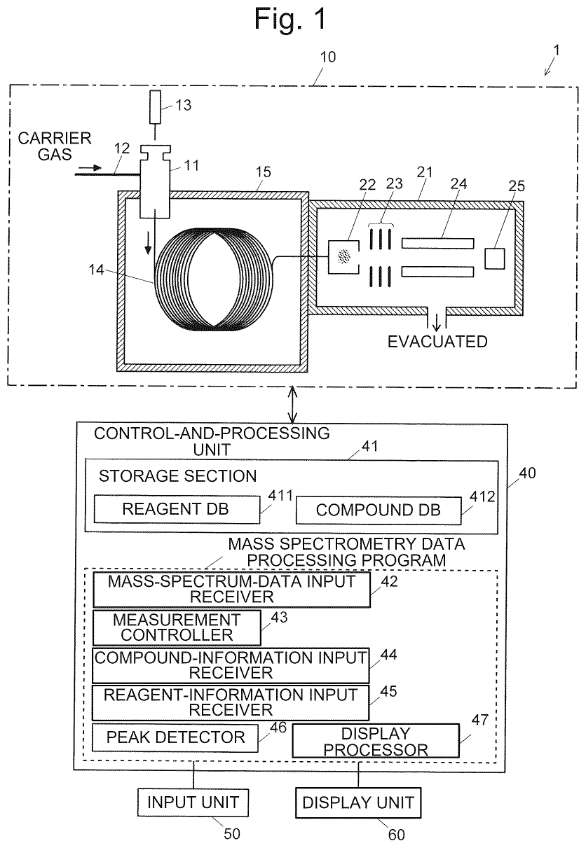

FIG. 1 shows the configuration of the main components of a gas chromatograph mass spectrometry system 1 according to the present embodiment. The gas chromatograph mass spectrometry system 1 includes a gas chromatograph mass spectrometry unit 10 and a control-and-processing unit 40. The control-and-processing unit 40 corresponds to one embodiment of the device for processing mass spectrometry data according to the present invention.

The gas chromatograph mass spectrometry unit 10 includes a gas chromatograph section configured to separate compounds contained in a sample and a mass spectrometer section configured to perform a mass spectrometric analysis of the compounds separated by the gas chromatograph section.

The gas chromatograph section includes a sample vaporization chamber 11, carrier gas passage 12, micro syringe 13, column 14 and column oven 15. The carrier gas passage 12 is connected to the sample vaporization chamber 11. A carrier gas, such as helium, supplied from a gas source (not shown) is introduced through the carrier gas passage 12 into the sample vaporization chamber 11. A sample introduced from the micro syringe 13 is vaporized within the sample vaporization chamber 11 and carried into the column 14 by the stream of the carrier gas. The temperature of the column 14 is controlled to be a predetermined temperature by the column oven 15. While the sample is flowing through the column 14, the compounds in the sample are separated from each other. Although the present embodiment uses the micro syringe 13 as the sample introduction unit, a different type of device may also be used for introducing the sample.

The mass spectrometer section includes a vacuum chamber 21 evacuated to a predetermined degree of vacuum by a vacuum pump (not shown). An ion source 22, ion lens 23, quadrupole mass filter 24 and ion detector 25 are arranged within the vacuum chamber 21. The compounds separated from each other in the column 14 of the gas chromatograph section are sequentially ionized in the ion source 22. After being converged into a specific flight direction by the ion lens 23, the ions are mass-separated by the quadrupole mass filter 24 and detected by the ion detector 25. For example, an electron ionization source or chemical ionization source is used as the ion source 22.

The control-and-processing unit 40 includes a storage section 41. The storage section 41 holds a reagent database 411 in which information concerning derivatization reagents described later (such as the reagent name, reagent ID, molecular formula and molecular structure) is related to the mass-to-charge ratios and structural formulae of the ions that will be generated from the respective reagents. The storage section 41 also holds a compound database (library) 412 in which information concerning various kinds of known compounds (such as the compound name, molecular formula, molecular structure, retention time, mass spectrum and calibration curve) is recorded. Although the reagent database 411 and the compound database 412 in the present embodiment are stored in the storage section 41, similar databases accessible through the Internet or similar communication networks may also be used.

The control-and-processing unit 40 includes, as its functional blocks, a mass-spectrum-data input receiver 42, measurement controller 43, compound-information input receiver 44, reagent-information input receiver 45, peak locator 46 and display processor 47. The control-and-processing unit 40 is actually a common type of personal computer, for example, with the aforementioned functional blocks embodied by executing a pre-installed mass spectrometry data processing program on a processor. Additionally, an input unit 50 and a display unit 60 are connected to the control-and-processing unit 40.

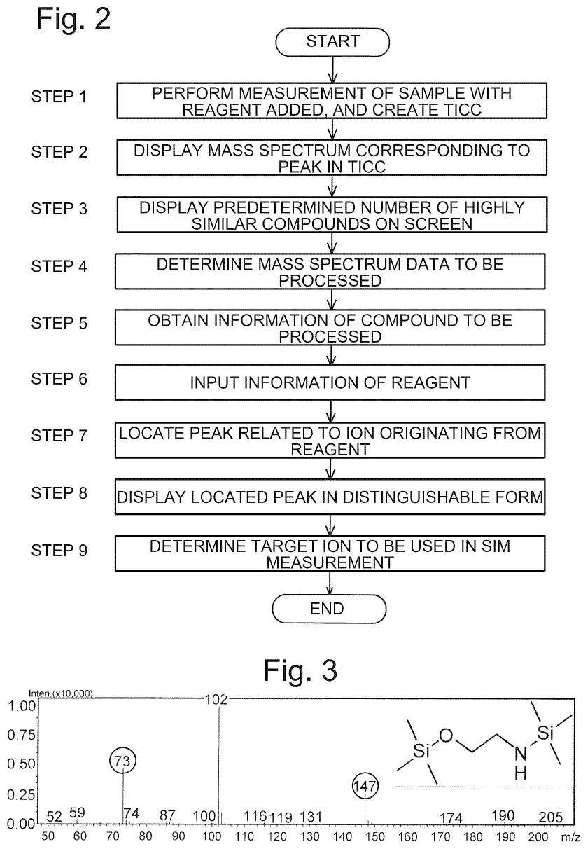

Next, the flow of an analysis using the gas chromatograph mass spectrometry system 1 according to the present embodiment is described with reference to the flowchart of FIG. 2. The following description deals with the case of determining a target ion to be used for the qualitative and/or quantitative determination of a metabolite contained in a sample.

When the quantity of a metabolite contained in a sample is to be determined, an SIM or MRM measurement is performed. In the case of using a mass spectrometer unit having a single mass filter as in the present embodiment, an SIM measurement is performed. When a so-called tandem type of mass spectrometer unit having a front mass filter and a rear mass filter is used, an MRM measurement can also be performed. In the SIM measurement, a target ion is previously designated for each metabolite which is a target of the measurement, and a calibration curve is created from the area value or intensity value of a peak acquired by a measurement of a standard sample of the metabolite. In an actual measurement, the quantity is determined from the measured value of the area or intensity of the peak of the target ion with reference to the calibration curve. In the SIM measurement (and also the MRM measurement) of a metabolite, a mass spectrum is acquired by a scan measurement, and a target ion is designated from the ions detected in that spectrum.

When the user has issued a command to initiate the analysis, the mass-spectrum-data input receiver 42 determines the mass spectrum data to be used for the data analysis.

A description of this measurement data is as follows: In many cases, the metabolites contained in biological samples are low in volatility and difficult to vaporize within the sample vaporization chamber 11 of the gas chromatograph section. Accordingly, for the measurement of such a metabolite, a pretreatment for improving the volatility of the metabolite is performed using various derivatization reagents. One example of this pretreatment is the process of increasing the volatility of the metabolite by trimethylsilylation, i.e., the derivatization of the metabolite through the addition of a trimethylsilyl group (Si(CH₃)₃) to the metabolite in the sample. The present example assumes that the metabolite contained in the sample is trimethylsilylated for the measurement.

The measurement data is acquired by the measurement controller 43 by operating the gas chromatograph mass spectrometer section 10 as follows: After setting the pretreated sample in the micro syringe 13, the user issues a command for initiating the measurement, whereupon the measurement controller 43 supplies the carrier gas through the carrier gas passage 12 to the sample vaporization chamber 11 at a predetermined flow rate and injects a predetermined amount of sample from the micro syringe 13 into the sample vaporization chamber 11. The sample injected into the sample vaporization chamber 11 is vaporized within the same chamber 11 and carried into the column 14 by the stream of the carrier gas. Within the column 14, the compounds contained in the sample gas are separated from each other.

The compounds in the sample separated by the column 14 are sequentially ionized in the ion source 22 of the mass spectrometer section and converged into a narrow flight path by the ion lens 23 before entering the quadrupole mass filter 24. A predetermined voltage for performing a scan of the mass-to-charge ratio of the ion which is allowed to pass through is applied to the quadrupole mass filter 24. At each point in time, an ion having a mass-to-charge ratio corresponding to the voltage applied at that point in time is allowed to pass through the quadrupole mass filter 24 and be detected by the ion detector 25. The output signals from the ion detector 25 are sequentially stored in the storage section 41.

After the completion of the measurement, the mass-spectrum-data input receiver 42 retrieves the output signals of the ion detector 25 from the storage section 41 and creates a total ion current chromatogram (TICC; Step 1). Specifically, three-dimensional data showing the intensity value with respect to the two axes of time and mass-to-charge ratio is prepared from the output signals acquired by the measurement, and the TICC is created by totaling the intensity values in the direction of the mass-to-charge ratio in the three-dimensional data.

The user performs an operation for instructing the device to read the data created in the previously described manner and display the TICC on the screen of the display unit 60, followed by an operation for indicating a peak in the TICC. Then, the mass-spectrum-data input receiver 42 displays, on the screen of the display unit 60, a mass spectrum acquired within the period of time corresponding to the indicated peak (Step 2). It also compares that spectrum data with mass spectra of various compounds recorded in the compound database 412 stored in the storage section 41, and displays, on the screen of the display unit 60, a predetermined number of compound names and the mass spectra of the compounds concerned in descending order of their degrees of similarity (Step 3). The degree of similarity of a mass spectrum is determined by a calculation based on the positions and intensities of the peaks in the mass spectrum. The specific process of that calculation has been conventionally known, and therefore, no detailed description of the calculation will be given.

The user checks the predetermined number of compound names and the mass spectra of the compounds displayed on the screen of the display unit 60. After confirming that the metabolite which is a target of the measurement (which is hereinafter called the “target metabolite”) is included in those compounds, the user selects that metabolite. The mass-spectrum-data input receiver 42 receives the input of the metabolite by the user and designates that metabolite as a target of the subsequent processing. It also designates, as a target of the subsequent processing, the mass spectrum data created from the measurement data of the sample and used for the previously described comparing process (Step 4).

The previously described step of confirming that the mass spectrum corresponding to a peak in the TICC acquired by the measurement corresponds to the target metabolite guarantees that the mass spectrum to be used in the subsequent data analysis is the mass spectrum obtained by a correct measurement of the target metabolite. When the target of the measurement is a metabolite originating from a living organism or requiring a pretreatment as in the present embodiment, a peak of another compound similar to yet different from the target metabolite, or a peak of another compound generated in the pretreatment, may possibly appear in the TICC. If any one of these peaks is incorrectly selected by the user and a mass spectrum corresponding to that peak is used in the subsequent data analysis, an ion of a compound which is not the target metabolite will be designated as the target ion. Therefore, in the present embodiment, the degree of similarity with the mass spectra recorded in the compound database 412 is checked in the previously described manner.

When the user has selected the target metabolite from the predetermined number of compounds displayed on the screen of the display unit 60, the compound-information input receiver 44 obtains (receives an input of) information of the selected metabolite (compound) from the compound database 412, where the information includes at least the compound name and the molecular weight (Step 5).

Subsequently, the reagent-information input receiver 45 allows the user to input information of the derivatization reagent (in the present example, trimethylsilyl) used for the measurement by which the mass spectrum data was acquired (Step 6). For example, this input may be performed in such a manner that the reagent-information input receiver 45 displays, on the screen of the display unit 60, a list of the reagents recorded in the reagent database 411 stored in the storage section 41 and allows the user to select the reagent used in the measurement from the list. When a plurality of reagents were used, the reagent-information input receiver 45 should allow the user to input information of all of the used reagents. If the reagent used in the measurement by the user is not recorded in the reagent database 411 stored in the storage section 41, the reagent-information input receiver 45 prompts the user to input information of the used derivatization reagent (e.g., the reagent name, reagent ID, molecular formula and molecular structure) as well as the mass-to-charge ratios of the ions which will be generated from that reagent. The information thus inputted is added to the reagent database 411.

After the input of the reagent information, the peak locator 46 reads the information concerning the inputted reagent from the reagent database 411 and compares the mass-to-charge ratios of the peaks included in the mass spectrum data with those of the ions which will be generated from that reagent, to locate the matching mass-to-charge ratios (Step 7).

After a peak or peaks have been located by the peak locator 46, the display processor 47 creates a mass spectrum for the screen display from the mass spectrum data and shows that mass spectrum on the screen of the display unit 60 in a form in which the peak or peaks located by the peak locator 46 are distinguishable from the other peaks among the peaks in the mass spectrum (Step 8). The analysis operator refers to the mass spectrum shown on the display unit 60 and designates a target ion in the SIM measurement (Step 9).

FIGS. 3-7 are display examples of the mass spectrum by the display processor 47. The present mass spectrum was obtained by a gas chromatograph mass spectrometric analysis of trimethylsilylated 2-Aminoethanol. Shown in the upper-right area of the mass spectrum in FIGS. 3-7 is the structure of a molecule in which two trimethylsilyl groups (TMS) are added to 2-Aminoethanol. Although each of the display examples of FIGS. 3-7 is hereinafter individually described, it is possible to adopt a display mode in which two or more of those examples are combined.

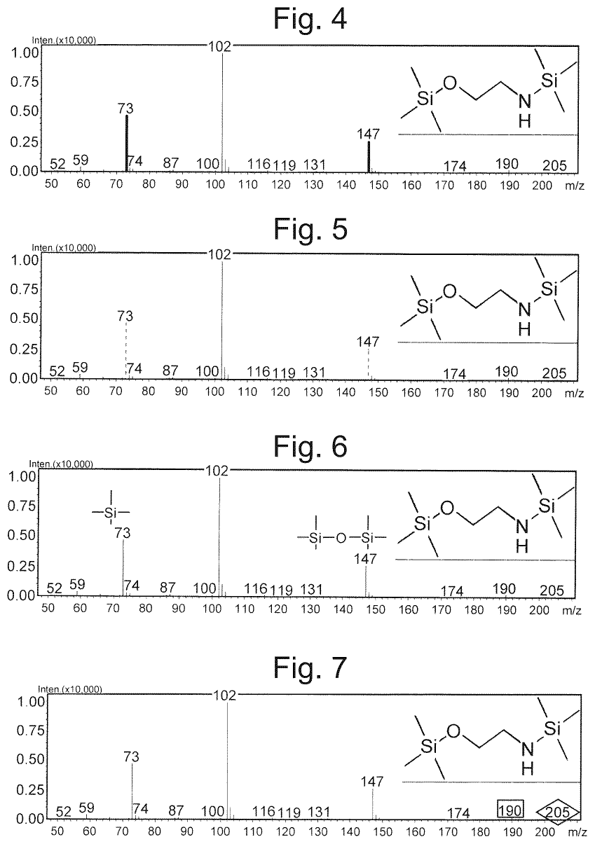

In the example of FIG. 3, each of the peaks of two ions (m/z=73 and 147) originating from TMS among the peaks in the mass spectrum is highlighted by a circle placed on the value of the mass-to-charge ratio shown above the peak. Although the present drawing is monochromatic, an appropriate color may be used for the highlighting (the same also applies in the following FIGS. 4-7). Such a display mode enables an individual who is not skilled in the analysis to easily recognize the peaks of the ions originating from TMS and designate an ion corresponding to one of the other peaks as the target ion in the SIM measurement.

In the example of FIG. 4, each of the peaks of two ions (m/z=73 and 147) originating from TMS among the peaks in the mass spectrum is highlighted by a thick line. Similar to the previous example, the present display mode enables the user to easily recognize the peaks of the ions originating from TMS and designate an ion corresponding to one of the other peaks as the target ion in the SIM measurement.

In the example of FIG. 5, each of the peaks of two ions (m/z=73 and 147) originating from TMS among the peaks in the mass spectrum is shown in an obscure form by a thin broken line. This display mode makes the peaks other than those originating from TMS more noticeable on the display. Therefore, the user can designate an ion corresponding to one of those peaks as the target ion in the SIM measurement.

In the example of FIG. 6, each of the peaks of two ions (m/z=73 and 147) originating from TMS among the peaks in the mass spectrum has its structural formula shown above the peak. This display mode allows the user to easily recognize the peaks of the ions originating from TMS as in the previous examples. Furthermore, it also allows the user to understand which component of the TMS the ion corresponds to.

In the example of FIG. 7, the peaks of two ions having the largest and second largest mass-to-charge ratios among the peaks in the mass spectrum are respectively indicated by rhombic and rectangular marks placed on the values of the mass-to-charge ratios shown above the two peaks.

In a gas chromatograph mass spectrometric analysis like the present embodiment, a molecular ion of the derivatized compound normally appears as a peak of the ion having the largest mass-to-charge ratio in the mass spectrum. Accordingly, in the example shown in FIG. 7, it is easy to understand that the ion of m/z=205 denoted by the rhombic mark is the molecular ion. Additionally, in a mass spectrum acquired by a measurement of a compound which has been pretreated for trimethylsilylation as in the present embodiment, the ion having the second largest mass-to-charge ratio often results from the removal of the methyl group from the molecular ion. The example shown in FIG. 7 is also such a case; the second largest mass-to-charge ratio is m/z=190 and its difference from the mass-to-charge ratio of the molecular ion is 15, which corresponds to the methyl group. These ions having large mass-to-charge ratios are either the molecular ion itself or an ion resulting from an insignificant fragmentation of the molecular ion. As compared to fragment ions which are small fragments of the molecular ion, the aforementioned ions have a greater amount of structural information, and therefore have a higher degree of identifiability. Accordingly, by using one of the highlighted ions as the target ion in the SIM measurement, the analysis operator can perform an accurate quantitative determination with a high level of identifiability. The process of locating these peaks may also be performed by the peak locator 46.

Additionally, in the example of FIG. 7, it is preferable that the peak locator 46 be further configured to verify the mass-to-charge ratio of the ion having the largest mass-to-charge ratio. Specifically, in the example of FIG. 7, the molecular weight of the original compound, 2-Aminoethanol, is 61, and that of TMS is 73. Since there are two TMSs added, the verifying calculation is 61+73×2−2=205, which confirms that the peak of the ion having the largest mass-to-charge ratio is an ion consisting of one 2-Aminoethanol with two TMSs added. It should be noted that the term “−2” in the previous calculation means the subtraction of the atomic weight of two hydrogen atoms (H) replaced by the TMSs.

Although the description so far has been concerned with the case of determining a target ion in an SIM measurement, the previously described configuration can also be similarly applied in the case of determining a target ion in an MRM measurement. Specifically, information related to the precursor ion and product ions originating from a reagent can be stored in the storage section 41, and a peak or peaks matching that information can be indicated in such a manner that they are distinguishable from other peaks in a mass spectrum which is displayed when the user selects a precursor ion for an MS/MS scan measurement.

The previously described configuration can also be suitably used for a qualitative determination of a compound contained in a sample. In a qualitative determination of a compound contained in a sample, when a mass spectrum acquired by a measurement of the sample as in the previous embodiment is compared with the mass spectra of the known compounds recorded in the compound database 412, a plurality of candidate compounds which are close to each other in degree of similarity may possibly be extracted. A skilled analysis operator will be able to exclude peaks originating from the reagent and select an appropriate compound based on the degree of similarity of the other peaks. In contrast, an analysis operator who is not skilled in the analysis may possibly make a false-positive identification by selecting a compound based on the similarity of the peaks of the ions which originate from the reagent.

On the other hand, in the present embodiment, since the peaks of the ions originating from the reagent are displayed in such a manner that they are distinguishable from the other ions, it is possible to select an appropriate compound and correctly perform the qualitative determination by giving lower priorities to candidate compounds having high degrees of similarity with the peaks of the ions originating from the reagent while giving higher priorities to candidate compounds having high degrees of similarity with the other peaks, or by referring to other indices (e.g., retention indices).

The previous embodiment is a mere example and can be appropriately changed or modified without departing from the spirit of the present invention.

In the previous embodiment, the input of the reagent information is performed after the input of the mass spectrum data acquired by the measurement and the compound information. The order of those inputs may be reversed. That is to say, the input of the mass spectrum data and the compound information may be performed after the input of the reagent information. The reason for inputting the compound information in the previous embodiment is to perform the calculation for verifying that the ion having the largest mass-to-charge ratio is the molecular ion. If this calculation is unnecessary, only the reagent information needs to be inputted.

The previous embodiment is the combination of a gas chromatograph and a single quadrupole mass spectrometer. Combining a gas chromatograph with a mass spectrometer having another configuration (e.g., a tandem quadrupole or time-of-flight configuration) is also possible. A liquid chromatograph may be used in place of the gas chromatograph. Furthermore, the previously described configuration can be similarly adopted in the case of using a stand-alone mass spectrometer without a chromatograph.

Although the previous embodiment was concerned with the case where trimethylsilyl (TMS) is used as the derivatization reagent for increasing the volatility of a metabolite which is a target of the measurement, the previously described configuration can be similarly adopted in the case of using other kinds of derivatization reagents. Furthermore, the previously described configuration can be similarly adopted in the case of using a reagent for a purpose other than an increase in volatility, as in the case of using a reagent for improving the ionization efficiency in the mass spectrometer.

### Modes

It is evident to a person skilled in the art that the previously described illustrative embodiment is a specific example of the following modes of the present invention.

### Clause 1

A device for processing mass spectrometry data according to one mode of the present invention includes:
a storage section which holds information in which reagent identification information identifying a reagent is related to the mass-to-charge ratio of an ion to be generated from the reagent;
a display section;
a mass-spectrum-data input receiver configured to receive an input of mass spectrum data acquired by a mass spectrometric analysis of a sample;
a reagent-information input receiver configured to receive an input of the reagent identification information identifying a reagent added to the sample in the mass spectrometric analysis;
a peak locator configured to compare the reagent identification information received by the reagent-information input receiver with the information stored in the storage section, and to locate, among the peaks included in the mass spectrum data received by the mass-spectrum-data input receiver, a peak associated with an ion generated from the reagent; and
a display processor configured to create a mass spectrum from the mass spectrum data, and to display, on the display section, the mass spectrum in such a manner that the peak located by the peak locator is shown in a form distinguishable from other peaks.

In the device for processing mass spectrometry data according to Clause 1, when an analysis operator has entered mass spectrum data acquired by a mass spectrometric analysis of a sample as well as reagent identification information which identifies the reagent added to the sample, the device creates a mass spectrum from the mass spectrum data and displays it on the display section in a form in which a peak associated with an ion originating from the reagent is distinguishable from other peaks. Therefore, the user can easily recognize a peak associated with an ion originating from the reagent in the mass spectrum, regardless of the level of skill of the user.

### Clause 2

In a device for processing mass spectrometry data according to Clause 2, which is one mode of the device for processing mass spectrometry data according to Clause 1, the display processor is configured to draw the peak located by the peak locator and other peaks using lines having different thicknesses and/or colors.

The device for processing mass spectrometry data according to Clause 2 allows users to visually recognize a peak associated with an ion originating from a reagent, based on the thickness and/or color of the drawing line of each peak in the mass spectrum displayed on the screen of the display unit. For example, the peak of an ion originating from a reagent can be shown by a thick line or in a noticeable color so as to caution the analysis operator against selecting this peak as a target ion in an SIM or MRM measurement. Conversely, the peak of an ion originating from a reagent can be shown by a thin line or in an obscure color to make the other peaks more noticeable so that the analysis operator will be urged to select a target ion from these peaks. In the case of performing a qualitative determination of a compound by comparing measured mass spectrum data with those recorded in a compound database, the device can help users to select an appropriate compound and correctly perform the qualitative determination, by giving lower priorities to candidate compounds having high degrees of similarity with the peaks of the ions originating from the reagent while giving higher priorities to candidate compounds having high degrees of similarity with the other peaks, or by referring to other indices (e.g., retention indices).

### Clause 3

In a device for processing mass spectrometry data according to Clause 3, which is one mode of the device for processing mass spectrometry data according to Clause 1 or 2, the display processor is configured to display, in the vicinity of each peak in the mass spectrum, the mass-to-charge ratio of the peak in such a manner that the mass-to-charge ratio of the peak located by the peak locator is distinguishable from the mass-to-charge ratios of other peaks.

The device for processing mass spectrometry data according to Clause 3 allows users to recognize a peak associated with an ion originating from a reagent along with the mass-to-charge-ratio information of that ion.

### Clause 4

In a device for processing mass spectrometry data according to Clause 4, which is one mode of the device for processing mass spectrometry data according to one of Clauses 1-3, the display processor is configured to display, in a highlighted form, the peak having the largest mass-to-charge ratio and/or the mass-to-charge ratio of that peak among the peaks in the mass spectrum.

For example, in the case of a mass spectrum acquired by a gas chromatograph mass spectrometric analysis, a molecular ion will normally be the ion having the largest mass-to-charge ratio. The device for processing mass spectrometry data according to Clause 4 displays the peak of this ion and/or its mass-to-charge ratio in a highlighted form in order to present the molecular ion to the analysis operator as a candidate of a target ion in an SIM or MRM measurement. When a qualitative determination of a compound is performed, the qualitative determination can be correctly performed by putting emphasis on the peak of the molecular ion having the largest mass-to-charge ratio.

### Clause 5

A device for processing mass spectrometry data according to Clause 5, which is one mode of the device for processing mass spectrometry data according to Clause 4, further includes:
a compound-information input receiver configured to receive an input of information of the mass-to-charge ratio of a compound contained in the sample,
where the peak locator is configured to determine the mass-to-charge ratio of an ion generated by a reaction of the compound and the reagent, using the mass-to-charge ratio of the ion generated from the reagent corresponding to the reagent identification information received by the reagent-information input receiver and the information of the mass-to-charge ratio of the aforementioned compound.

The device for processing mass spectrometry data according to Clause 5 can compare the mass-to-charge ratio calculated by the peak locator with the information of the mass-to-charge ratio of the ion corresponding to the peak highlighted by the display processor.

### Clause 6

In a device for processing mass spectrometry data according to Clause 6, which is one mode of the device for processing mass spectrometry data according to Clause 4 or 5:

the reagent is a reagent for derivatizing the compound contained in the sample by adding a trimethylsilyl group to the compound; and the display processor is further configured to display, in a highlighted form, the peak having the second largest mass-to-charge ratio and/or the mass-to-charge ratio of that peak among the peaks in the mass spectrum.

In a mass spectrum acquired by a measurement of a compound subjected to a derivatizing pretreatment which includes adding a trimethylsilyl group (trimethylsilylation), the peak having the second largest mass-to-charge ratio corresponds to an ion resulting from the removal of a methyl group from the molecular ion generated by a reaction of the compound and the reagent. This ion has a high degree of identifiability due to its structural information being similar to that of the molecular ion. The device for processing mass spectrometry data according to Clause 6 can display the molecular ion and the ion resulting from the removal of the methyl group from the molecular ion in a highlighted form, thereby presenting those ions to the analysis operator as candidates of the target ion in an SIM or MRM measurement. In the case of performing a qualitative determination of a compound by comparing measured mass spectrum data with those recorded in a compound database, the device can more correctly perform the qualitative determination by putting emphasis on the degree of similarity of those ions.

### REFERENCE SIGNS LIST

1 . . . Gas Chromatograph Mass Spectrometry System
10 . . . Gas Chromatograph Mass Spectrometer Section
11 . . . Sample Vaporization Chamber
12 . . . Carrier Gas Passage
13 . . . Micro Syringe
14 . . . Column
15 . . . Column Oven
21 . . . Vacuum Chamber
22 . . . Ion Source
23 . . . Ion Lens
24 . . . Quadrupole Mass Filter
25 . . . Ion Detector
40 . . . Control-and-Processing Unit
41 . . . Storage Section
411 . . . Reagent Database
412 . . . Compound Database
42 . . . Mass-Spectrum-Data Input Receiver
43 . . . Measurement Controller
44 . . . Compound-Information Input Receiver
45 . . . Reagent-Information Input Receiver
46 . . . Peak Locator
47 . . . Display Processor
50 . . . Input Unit
60 . . . Display Unit

1. A device for processing mass spectrometry data, comprising:
a storage section which holds information in which reagent identification information identifying a reagent is related to a mass-to-charge ratio of an ion to be generated from the reagent;
a display section;
a mass-spectrum-data input receiver configured to receive an input of mass spectrum data acquired by a mass spectrometric analysis of a sample;
a reagent-information input receiver configured to receive an input of the reagent identification information of a reagent added to the sample in the mass spectrometric analysis;
a peak locator configured to compare the reagent identification information received by the reagent-information input receiver with the information stored in the storage section, and to locate, among peaks included in the mass spectrum data received by the mass-spectrum-data input receiver, a peak associated with an ion generated from the reagent; and
a display processor configured to create a mass spectrum from the mass spectrum data, and to display, on the display section, the mass spectrum in such a manner that the peak located by the peak locator is shown in a form distinguishable from other peaks.

2. The device for processing mass spectrometry data according to claim 1, wherein the display processor is configured to draw the peak located by the peak locator and other peaks using lines having different thicknesses and/or colors.

3. The device for processing mass spectrometry data according to claim 1, wherein the display processor is configured to display, in a vicinity of each peak in the mass spectrum, a mass-to-charge ratio of the peak in such a manner that the mass-to-charge ratio of the peak located by the peak locator is distinguishable from mass-to-charge ratios of other peaks.

4. The device for processing mass spectrometry data according to claim 1, wherein the display processor is configured to display, in a highlighted form, a peak having a largest mass-to-charge ratio and/or a mass-to-charge ratio of that peak among the peaks in the mass spectrum.

5. The device for processing mass spectrometry data according to claim 4, further comprising:
a compound-information input receiver configured to receive an input of information of a mass-to-charge ratio of a compound contained in the sample,
wherein the peak locator is configured to determine a mass-to-charge ratio of an ion generated by a reaction of the compound and the reagent, using the mass-to-charge ratio of the ion generated from the reagent corresponding to the reagent identification information received by the reagent-information input receiver and the information of the mass-to-charge ratio of the aforementioned compound.

6. The device for processing mass spectrometry data according to claim 4, wherein:
the reagent is a reagent for derivatizing a compound contained in the sample by adding a trimethylsilyl group to the compound; and
the display processor is further configured to display, in a highlighted form, a peak having a second largest mass-to-charge ratio and/or a mass-to-charge ratio of that peak among the peaks in the mass spectrum.
